(19) United States
(12) Patent Application Publication
(10) Pub. No.: US 2022/0079740 A1
(43) Pub. Date: Mar. 17, 2022

(54) ORTHOPEDIC ANCHOR AND SURGICAL TOOL INTEGRATED WITH ANCHOR FASTENING MEMBER

(71) Applicant: AJU PHARM CO., LTD., Seoul (KR)

(72) Inventors: Jae Young SONG, Seoul (KR); Jin Kwon LEE, Seoul (KR)

(73) Assignee: AJU PHARM CO., LTD., Seoul (KR)

(21) Appl. No.: 17/424,023

(22) PCT Filed: May 25, 2019
(86) PCT No.: PCT/KR2019/006291

§ 371 Date: Jul. 19, 2021

(30) Foreign Application Priority Data
Jan. 21, 2019 (KR) 10-2019-0007738

(51) Int. Cl. A61F 2/08

(52) U.S. Cl. CPC A61F 2/0811; A61F 2/0805; A61F 2002/0888; A61F 2002/0858; A61F 2002/0817

(57) ABSTRACT

A surgical tool having an integrated orthopedic anchor and an anchor coupler are proposed. When an anchor (100) is coupled and fixed to an anchor coupling portion (220) of an anchor coupler (200), arc recesses (130a) formed in through-holes (130) and arc recesses (221a) formed at an anchor coupling portion (220) are fitted to each other, so suture insertion holes (20) aligned with holes in the anchor coupling portion (220) and having wide inlets are formed and a suture (10) may be easily connected. Further, when the anchor coupler (200) is rotated and pressed in surgery, the protruding front ends (221) separated at the anchor coupling portion (220) and protruding through the through-holes (130) at both sides of the front end of the anchor body (110) bore a bone tissue while covering an outer surface of the front end portion of the anchor (100).

### TECHNICAL FIELD

The present disclosure relates to a surgical tool in which an anchor to which a suture for surgery is connected and an anchor coupler for firmly coupling and fixing the anchor in a bone tissue are integrated.

### BACKGROUND ART

It is necessary in the medical community to introduce and develop minimal invasive surgery and robot surgery to quickly cure patients in the field of orthopedics.

In the field of orthopedics, anthroscopic surgery is currently increasingly applied to damaged ligaments or ruptured muscles due to a sports injury, arthritis, or degenerative diseases and will be much popularized.

Accordingly, anchors (suture anchor) are used to bond injured joint capsule, cartilage, muscle, and ligament.

The surgery process involves finely incising a diseased part or checking the diseased part through arthroscopy, fixing the muscle or ligament of the diseased part with a suture (e.g., a fiber-wire suture), expose the diseased part, and then inserting a screw (anchor) with a thread into the diseased part.

A suture is attached to the anchor, so a separated ligament or muscle can be sewn and bonded to a bone. In this case, the anchor should be inserted through a cannula.

A separated or ruptured ligament is pulled and bonded to a bone by the suture attached to the anchor and then a knot is made.

Since this knot should be made outside an endoscope, it takes a considerable time to make the knot. Further, if the knot is accurately made, the suture is untied or loosened, so the muscle or ligament may not be bonded to the bone.

### DOCUMENTS OF RELATED ART

### Patent Document

(Patent Document 1) KR10-1429354B1

(Patent Document 2) KR10-2015-0144047 A

(Patent Document 3) KR10-2014-0116211 A

(Patent Document 4) KR10-2015-0127233 A

### DISCLOSURE

### Technical Problem

An objective of the present disclosure is to provide a surgical tool having an integrated orthopedic anchor and anchor coupler in which the anchor can be easily fastened and fixed in advance to an anchor coupling portion of the anchor coupler.

Another objective of the present disclosure is to provide a surgical tool having an integrated orthopedic anchor and anchor coupler, in which when the anchor is coupled and fixed to an anchor coupling portion of the anchor coupler, arc grooves formed at the anchor and arc grooves formed at the anchor coupling portion are fitted to each other, suture insertion holes aligned with holes in the anchor coupling portion and having wide inlets are formed naturally formed, whereby a suture can be freely selectively connected and can be very easily connected.

Another objective of the present disclosure is to provide a surgical tool having an integrated orthopedic anchor and anchor coupler that is safe because, in surgery, both separate front end portions of the anchor coupling portion which protrude from both sides of the anchor bore a bone tissue and the anchor is thread-fastened, whereby the front end of the anchor and the both separated front end portions of the anchor coupling portion are not damaged during surgery.

Another objective of the present disclosure is to provide a high-quality surgical tool having an integrated orthopedic anchor and anchor coupler in which a dense thread portion having a pitch than the front portion is formed at the rear portion of the outer surface of the anchor, so a fastening portion by the dense thread portion is increased and the anchor is firmly thread-fastened without loosening, whereby a muscle or a ligament can be safely bonded to a bone by pulling a suture.

### Technical Solution

In order to achieve the objectives of the present disclosure, a surgical tool having an integrated orthopedic anchor and anchor coupler includes:

an anchor 100 having a cylindrical anchor body 110 that has threads 111 on an outer surface thereof,

an anchor coupling portion-insertion groove 120 that is formed in the cylindrical anchor body inward from a rear surface of the cylindrical anchor body 110, and

through-holes 130 that are formed through both sides of a front end of the anchor body 110 from the anchor coupling portion-insertion groove 120 to form a suture hook 131 and has arc recessions 130a formed on an inner surface thereof and angulated recessions 130b formed on an outer surface thereof; and

an anchor coupler 200 having an anchor coupling portion 220 that is formed at a front end of a hollow rod 210 to correspond to the anchor coupling portion-insertion groove 120 of the anchor 100 and is fitted in the anchor coupling portion-insertion groove 120,

protruding front end portions 221 that are laterally separated at a front of the anchor coupling portion 220, are inserted in the through-holes 130 at both sides of the front end of the anchor body 110 to protrude from the through-holes, have arc recessions 221a formed on inner surface thereof to face the arc recessions 130a formed on the inner surfaces of the through-holes 130 of the anchor body 110, and have angulated outer surfaces 221b formed on outer surface thereof to correspond to the angulated recessions 130b formed on the outer surfaces of the through-holes 130 of the anchor body 110, and

a handle 230 that is formed at a rear end of the hollow rod 210 to be used to rotate the rod 210 and has a suture holder 231 being able to fix an end of a suture 10 passing through the rod 210,

wherein the anchor and the anchor coupler are integrated; when the anchor 100 is coupled and fixed to the anchor coupling portion 220 of the anchor coupler 200, the arc recessions 130a formed at the through-holes 130 on both sides of the front end of the anchor body 110 and the arc recessions 221a formed at the anchor coupling portion 220 are fitted to each other, so suture insertion holes 20 aligned with holes in the anchor coupling portion 220 and having wide inlets are formed and the suture 10 is easily connected;

when the anchor coupler 200 is rotated and pressed in surgery, the protruding front ends 221 separated at the anchor coupling portion 220 and protruding through the through-holes 130 at both sides of the front end of the anchor body 110 bore a bone tissue while covering an outer surface of the front end portion of the anchor 100; and damage to the protruding front end portions 221 and the anchor 100 is prevented due to thread-fastening of the anchor 100.

Further, in the surgical tool having an integrated orthopedic anchor and anchor coupler according to an embodiment of the present disclosure, the protruding front ends 221 formed at both side of the front end of the anchor coupling portion 220 of the anchor coupler 200 may be formed to be pointed forward when seen from a side to easily bore a bone tissue 30.

In the surgical tool having an integrated orthopedic anchor and anchor coupler according to an embodiment of the present disclosure, the angulated outer surfaces 221b may be each formed in a “>” shape when the protruding front ends 221 formed at both side of the front end of the anchor coupling portion 220 of the anchor coupler 200 are seen from the front, so when the protruding front ends bore a bone tissue 30, the protruding front ends 221 may be smoothly pressed and inserted into the bone tissue 30 without excessively pressing the bone tissue 30 due to reduction of friction resistance against rotation, whereby the bone tissue 30 may not be damaged and the protruding front ends 221 may not be broken.

In the surgical tool having an integrated orthopedic anchor and anchor coupler according to an embodiment of the present disclosure, a dense thread portion 111a having a gap (pitch) smaller than the threads 111 at a front portion of the anchor body 110 of the anchor 100 is formed on the outer surface of a rear portion of the anchor body 110 so that a fastening force by the dense thread portion 111a is increased and the anchor 100 is firmly thread-fastened in a bone tissue without loosening.

### Advantageous Effects

According to the surgical tool having an integrated orthopedic anchor and anchor coupler according to an embodiment of the present disclosure, when the anchor is coupled and fixed to the anchor coupling portion of the anchor coupler, the arc recessions formed at the through-holes on both sides of the front end of the anchor body and the arc recessions formed at the anchor coupling portion are fitted to each other, so suture insertion holes aligned with holes in the anchor coupling portion and having wide inlets are naturally formed. Accordingly, it is possible to connect the suture first to the anchor and then couple the anchor with the suture connected thereto to the anchor coupler. Further, it is possible to couple the anchor to the anchor coupler and then connect the suture. That is, the suture can be freely selectively connected, and the suture can be very simply and quickly connected regardless of using any ways.

Further, according to the surgical tool having an integrated orthopedic anchor and anchor coupler according to an embodiment of the present disclosure, the protruding front end portions 221 laterally separated at the front end of the anchor coupling portion 220 protrude from the front end of the anchor. Accordingly, when a user rotates and presses the anchor coupler by holding and rotating the handle in surgery, the protruding front end portions first bore the bone tissue.

Further, since the anchor can be thread-fastened, the anchor can be very simply and quickly implanted. Further, particularly, since the protruding front ends are formed to be pointed forward and the angulated outer surfaces are each formed in a “>” shape when seen from the front, it is easy to bore a bone tissue and the protruding front ends are smoothly inserted into the bone tissue without excessively pressing the bone tissue due to reduction of friction resistance against rotation, whereby the bone tissue can be prevented from being damaged.

Further, according to the surgical tool having an integrated orthopedic anchor and anchor coupler according to an embodiment of the present disclosure, since excessive pressure is not applied to the surgical tool during surgery, damage to the both protruding front end portions such as breaking can be completely prevented.

Further, according to the surgical tool having an integrated orthopedic anchor and anchor coupler according to an embodiment of the present disclosure, since a dense thread portion having a gap (pitch) smaller than the threads at a front portion of the anchor body of the anchor is formed on the outer surface of a rear portion of the anchor body, when the anchor is thread-fastened in a bone tissue, the anchor is firmly thread-fastened without loosening, so it is possible to more safely bond a muscle or a ligament to a bone by pulling a suture connected to the anchor.

Therefore, since the surgical tool having an integrated orthopedic anchor and anchor coupler according to an embodiment of the present disclosure provides various advantages, as described above, the surgical tool is surely a very innovative invention that can be conveniently used.

### DESCRIPTION OF DRAWINGS

FIG. 1 is an entire perspective view showing an assembled state of a surgical tool having an integrated orthopedic anchor and anchor coupler according to an embodiment of the present disclosure;

FIG. 2 is a front exploded perspective view showing main parts of the surgical tool having an integrated orthopedic anchor and anchor coupler;

FIG. 3 is a rear exploded perspective view showing main parts of the surgical tool having an integrated orthopedic anchor and anchor coupler according to an embodiment of the present disclosure;

FIG. 4 is a partial cut view showing main parts of the surgical tool having an integrated orthopedic anchor and anchor coupler according to an embodiment of the present disclosure;

FIG. 5 is an exemplary cross-sectional view showing the assembled state of the surgical tool having an integrated orthopedic anchor and anchor coupler according to an embodiment of the present disclosure;

FIG. 6 is a front view showing the disassembled state of an anchor and an anchor coupler in the surgical tool having an integrated orthopedic anchor and anchor coupler according to an embodiment of the present disclosure;

FIG. 7 is a front enlarged view showing the assembled state of the anchor and the anchor coupler in the surgical tool having an integrated orthopedic anchor and anchor coupler according to an embodiment of the present disclosure;

FIG. 8 is a front view showing the state of the surgical tool having an integrated orthopedic anchor and anchor coupler according to an embodiment of the present disclosure before the anchor is implanted a bone tissue; and

FIG. 9 is a front view showing the state of the surgical tool having an integrated orthopedic anchor and anchor coupler according to an embodiment of the present disclosure after the anchor is implanted a bone tissue.

### REFERENCE NUMERALS

10: suture
20: suture insertion hole
100: anchor
110: anchor body
111: thread
111a: dense thread portion
120: anchor coupling portion-insertion groove
130: through-hole
130: through-hole
130a: arc recession
130b: angulated recession
131: suture hook
200: anchor coupler
210: rod
221: protruding front end portion
221a: arc recession
221b: angulated outer surface
230: handle
231: suture holder

### BEST MODE

Hereafter, embodiments of the present disclosure for achieving the objectives described above are described with reference to the accompanying drawings. The following embodiments are only example for helping understand the present disclosure and it should be understood that the present disclosure may be modified in various ways different from the embodiments described herein. However, in describing the present invention, detailed descriptions and drawings of well-known functions or components relating to the present disclosure will not be provided so as not to obscure the description of the present disclosure with unnecessary details. Further, the dimensions of some components are not shown with the actual scales and may be exaggerated in the drawings to help understand the present disclosure.

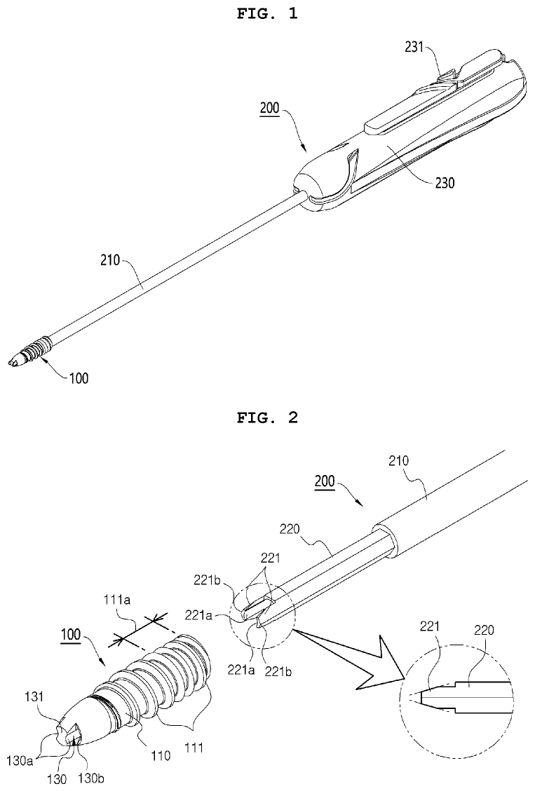

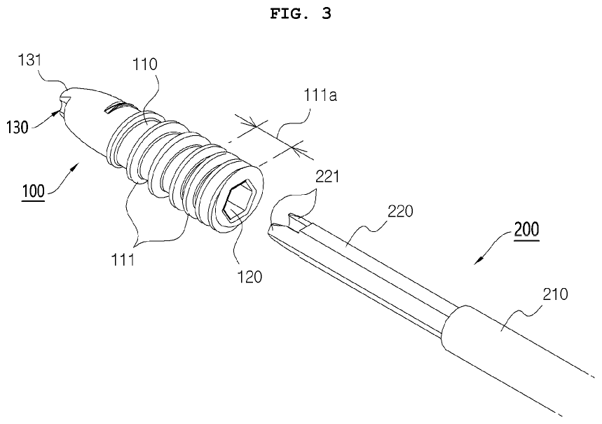

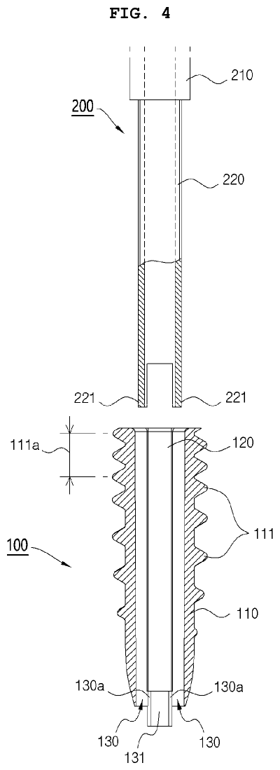

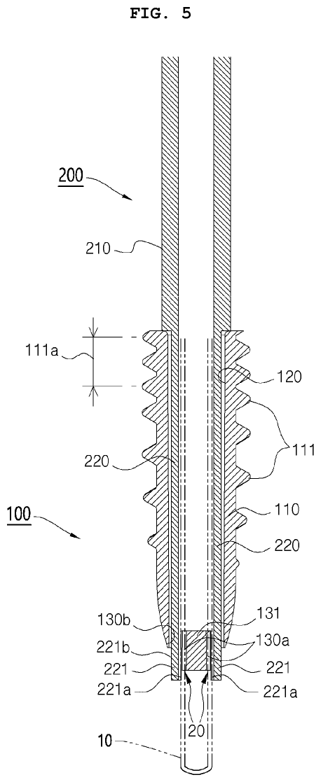

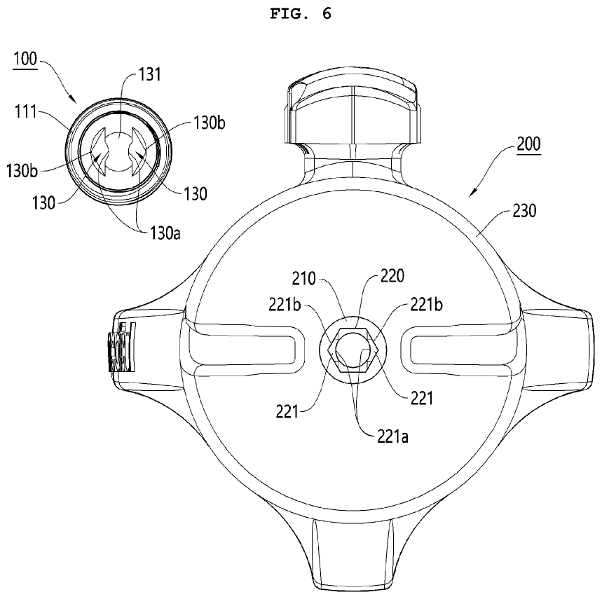

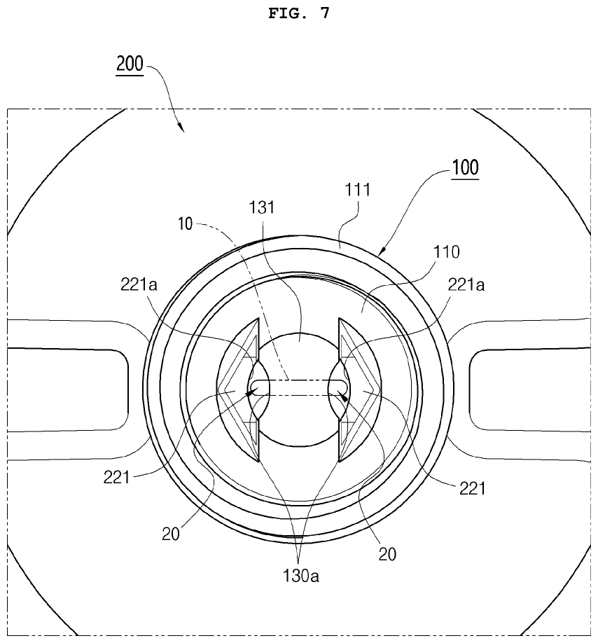

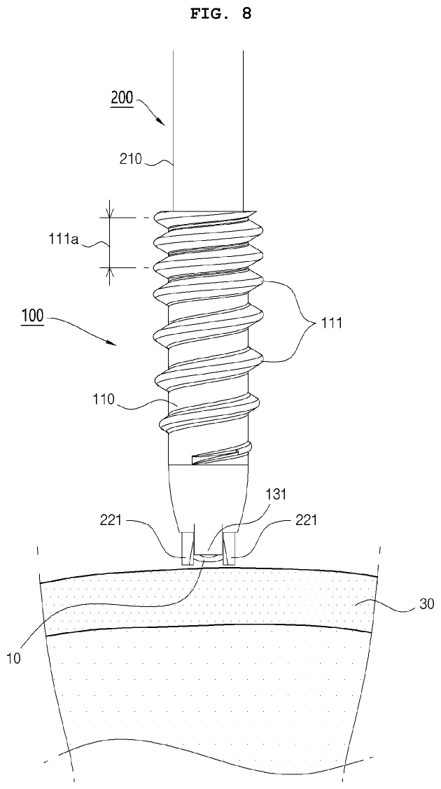

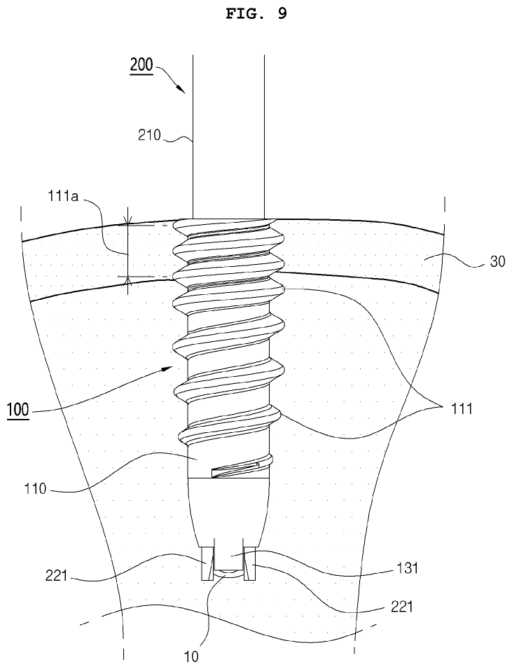

A surgical tool having an integrated orthopedic anchor and anchor coupler of the present disclosure, as shown in FIGS. 1 to 4, in a broad meaning, includes an anchor 100 that is thread-fastened and fixed in a bone tissue 30 with a suture 10 connected thereto, and an anchor coupler 200 that is used to thread-fasten the anchor 100 in a bone tissue by rotating the anchor 100.

The reason of integrating the orthopedic anchor and the anchor coupler is because the anchor and the anchor coupler discharge their duties in one-time use and cannot be reused due to sanitary reasons such as a possibility of infection.

First, the anchor 100 proposed herein is described.

An anchor body 110 of the anchor 100 has threads 111 on the outer surface.

The cylindrical anchor body 110 further has an anchor coupling portion-insertion groove 120 and the anchor coupling portion-insertion groove 120 is formed in the anchor body 110 inward from the rear surface of the anchor body 110.

Through-holes 130 are further formed in the cylindrical anchor body 110. The through-holes 130 are formed through two sides of the front end of the anchor body 110 from the anchor coupling portion-insertion groove 120, whereby a suture holder 131 is formed.

An arc recession 130a is formed on the inner surface of the through-hole and an angulated recession 130b is formed on the outer surface of the through-hole.

The anchor coupler 200 proposed herein is described hereafter.

The anchor coupler 200 has an anchor coupling portion 220 at the front end of a hollow rod 210. The anchor coupling portion 220 has a shape corresponding to the anchor coupling portion-insertion groove 120 of the anchor 100 so that torque can be transmitted to the anchor 100.

Both of the anchor coupling portion 220 and the anchor coupling portion-insertion groove 120 have a hexagonal shape in the present disclosure, but they are not limited thereto.

The anchor coupler 200 further has a pair of protruding front end portions 221 and the protruding front end portions 221 are laterally separated at the front of the anchor coupling portion 220.

The protruding front end portions 221 are cut to be slightly pointed forward when seen from a side.

The protruding front end portions 221 extending forward from the anchor coupling portion 220 are inserted in the through-holes 130 formed on both sides of the front end of the anchor body 110 such that both ends thereof protrude from the through-holes 130 to bore a bone tissue 30.

An arc recession 221 is formed on the inner surface of the protruding front end portion 221 to face the arc recession 130a formed on the inner surface of the through-hole 130 of the anchor body 110. Further, an angulated outer surface 221b is formed in a “>” shape on the outer surface of the protruding front end portion 221 to correspond to the angulated recession 131b on the outer surface of the through-hole 130.

Accordingly, since the protruding front end portions 221 are formed to be slightly pointed forward when seen from a side and the angulated outer surfaces 221b are each formed a “>” shape when seen from the front, the bone tissue 30 can be easily bored by the protruding front end portions 221. Further, since friction resistance against to rotation is decreased, the protruding front end portions 221 are smoothly inserted into the bone tissue 30 under pressure without excessively pressing the bone tissue 30, so damage to the bone tissue 30 is prevented.

Since excessive pressure is not applied, damage such as breakage of both protruding front end portions 221 is completely prevented.

Meanwhile, a handle 230 for rotating the hollow rod 210 is formed at the rear end of the rod 210. A suture holder 231 that can fix an end of a suture 10 connected to the anchor and passing through the rod 210 is formed in various shapes on the handle 230.

As described above, the surgical tool having an integrated orthopedic anchor and anchor coupler of the present disclosure has a structure having specific configuration.

Accordingly, when the anchor 100 is coupled and fixed to the anchor coupling portion 220 of the anchor coupler 200, the arc recessions 131a formed at the through-holes 130 on both sides of the front end of the anchor body 110 and the arc recessions 221a formed at the anchor coupling portion 220 are fitted to each other. Accordingly, suture insertion holes 20 aligned with holes in the anchor coupling portion 220 and having wide inlets are naturally formed.

Accordingly, it is possible to connect the suture 10 first to the anchor 100 and then couple the anchor 100 with the suture 10 connected thereto to the anchor coupler 200. Further, it is possible to couple the anchor 100 to the anchor coupler 200 and then connect the suture 10. That is, the suture 10 can be freely selectively connected, and the suture 10 can be very simply and quickly connected regardless of using any ways.

Further, according to the present disclosure, the protruding front end portions 221 laterally separated at the front end of the anchor coupling portion 220 are fitted to the through-holes 130 on both sides of the front end of the anchor body 110 such that only a portion of each of the front ends protrudes from the front end of the anchor 100. Accordingly, when a user rotates and presses the anchor coupler 200 by holding and rotating the handle 230 in surgery, the protruding front end portions 221 first bore the bone tissue 30. Further, since the anchor 100 can be thread-fastened, the anchor 100 can be very simply and quickly implanted.

Further, since only a portion of each of the front ends of the protruding front end portions 221 protrudes outward, the protruding front end portions 221 are formed to be slightly pointed forward when seen from a side, and the angulated outer surfaces 221b are formed a “>” shape when seen from the front, the bone tissue 30 can be easily bored. Further, since friction resistance against to rotation is decreased, the protruding front end portions 221 are smoothly inserted into the bone tissue 30 under pressure without excessively pressing the bone tissue 30, so damage to the bone tissue 30 can be prevented.

Further, since excessive pressure is not applied, damage such as breakage of both protruding front end portions 221 is completely prevented.

Meanwhile, a dense thread portion 111a having a gap (pitch) smaller than the threads at the front portion of the anchor body 110 of the anchor 100 are formed on the outer surface of the rear portion of the anchor body 110.

Accordingly, when the anchor 110 is thread-fastened in the bone tissue 30, the fastening force is increased by the dense thread portion 111a, so the anchor 100 is firmly thread-fastened in the bone tissue without loosening.

Therefore, it is possible to more safely bond a muscle or a ligament to the bone by pulling the suture 10 connected to the anchor.

Although the present disclosure was described with reference to limited exemplary embodiments and drawings, the present disclosure is not limited thereto and may be changed and modified in various ways within the spirit of the present disclosure and claims described below by those skilled in the art.

### INDUSTRIAL APPLICABILITY

The surgical tool having an integrated orthopedic anchor and anchor coupler according to an embodiment of the present disclosure can be used to firmly fix an anchor in a bone tissue in orthopedic surgery.

1. A surgical tool having an integrated orthopedic anchor and anchor coupler, the surgical tool comprising:
an anchor having a cylindrical anchor body that has threads on an outer surface thereof,
an anchor coupling portion-insertion groove that is formed in the cylindrical anchor body inward from a rear surface of the cylindrical anchor body, and
through-holes that are formed through both sides of a front end of the anchor body from the anchor coupling portion-insertion groove to form a suture hook and has arc recessions formed on an inner surface thereof and angulated recessions formed on an outer surface thereof; and
an anchor coupler having
an anchor coupling portion that is formed at a front end of a hollow rod to correspond to the anchor coupling portion-insertion groove of the anchor and is fitted in the anchor coupling portion-insertion groove,
protruding front end portions that are laterally separated at a front of the anchor coupling portion, are inserted in the through-holes at both sides of the front end of the anchor body to protrude from the through-holes, have arc recessions formed on inner surface thereof to face the arc recessions formed on the inner surfaces of the through-holes of the anchor body, and have angulated outer surfaces formed on outer surface thereof to correspond to the angulated recessions formed on the outer surfaces of the through-holes of the anchor body, and
a handle that is formed at a rear end of the hollow rod to be used to rotate the rod and has a suture holder being able to fix an end of a suture passing through the rod,
wherein the anchor and the anchor coupler are integrated; when the anchor is coupled and fixed to the anchor coupling portion of the anchor coupler, the arc recessions formed at the through-holes on both sides of the front end of the anchor body and the arc recessions formed at the anchor coupling portion are fitted to each other, so suture insertion holes aligned with holes in the anchor coupling portion and having wide inlets are formed and the suture is easily connected;
when the anchor coupler is rotated and pressed in surgery, the protruding front ends separated at the anchor coupling portion and protruding through the through-holes at both sides of the front end of the anchor body bore a bone tissue while covering an outer surface of the front end portion of the anchor; and
damage to the protruding front end portions and the anchor is prevented due to thread-fastening of the anchor.

2. The surgical tool of claim 1, wherein the protruding front ends formed at both side of the front end of the anchor coupling portion of the anchor coupler are formed to be pointed forward when seen from a side to easily bore a bone tissue.

3. The surgical tool of claim 1, wherein the angulated outer surfaces are each formed in a “>” shape when the protruding front ends formed at both side of the front end of the anchor coupling portion of the anchor coupler are seen from the front, so when the protruding front ends bore a bone tissue, the protruding front ends are smoothly inserted into the bone tissue without excessively pressing the bone tissue due to reduction of friction resistance against rotation, whereby the bone tissue is not damaged and the protruding front ends are not broken.

4. The surgery tool of claim 1, wherein a dense thread portion having a gap (pitch) smaller than the threads at a front portion of the anchor body of the anchor is formed on the outer surface of a rear portion of the anchor body so that a fastening force by the dense thread portion is increased and the anchor is firmly thread-fastened in a bone tissue without loosening.
